(19) United States
(12) Patent Application Publication
(10) Pub. No.: US 2022/0160915 A1
(43) Pub. Date: May 26, 2022

(54) CARD SANITIZING DEVICE

(71) Applicant: Tuan Quoc Dam, Round Rock, TX (US)

(72) Inventor: Tuan Quoc Dam, Round Rock, TX (US)

(21) Appl. No.: 17/531,789

(22) Filed: Nov. 21, 2021

Related U.S. Application Data
(60) Provisional application No. 63/116,315, filed on Nov. 20, 2020.

(51) Int. Cl. A61L 2/18

(52) U.S. Cl. CPC A61L 2/18; A61L 2202/16; A61L 2202/15; A61L 2202/122

(57) ABSTRACT

The present disclosure relates a device, a kit, and a method of sanitizing objects. The device includes a housing and a reservoir. The housing includes a first housing member and a second housing member, and a chamber disclosed therewithin. The first housing member and the second housing member are connected through a connector mechanism. The chamber is configured to receive a fluid-permeable sponge applicator therewithin. A reservoir containing a sanitizer fluid is disposed above the sponge applicator to wet the sponge applicator. An object is passed through a front opening and through the sponge applicator to be sanitized.

### RELATED APPLICATIONS

This application claims priority to provisional application serial no. 63116315 filed on Nov. 20, 2020, which is incorporated herein by reference in its entirety.

### FIELD OF INVENTION

The present disclosure relates generally to the field of sanitization. More particularly, the present disclosure relates to devices, kits, and methods for sanitization of objects.

### BACKGROUND OF THE INVENTION

A number of devices and methods for sanitizing objects are known in the art. Bacteria, germs, viruses, and the like can be harmful and/or dangerous (e.g., may transmit colds, flu, and/or other more serious illnesses such as COVID-19). While hand sanitizing gels are known in the art, even if used frequently, they may be relatively ineffective since even recently sanitized hands often grasp objects that may not have been sanitized.

Accordingly, improvements are sought in sanitizing personal objects such as credit cards, cellphone, keys and the like.

### SUMMARY OF THE INVENTION

While the way that the present invention addresses the disadvantages of the prior art will be discussed in greater detail below, in general, the present invention provides a device and a method for sanitizing objects at various places, such as at a point of sale.

One aspect of the invention features, in some embodiments, a multi-part housing retaining a sponge applicator and a sanitizer fluid reservoir above the sponge applicator. The housing provides access to fill the reservoir with a sanitizer fluid, which then drips onto and, in some cases, saturates the sponge applicator. In some cases, a saturated sponge applicator may be replaced as needed. Openings defined in the housing allow for a passage of an object along the sponge applicator to sanitize at least one face of the object. An exemplary use of the sanitizing device is to sanitize a card. The card may be passed in either direction through the housing, e.g., to facilitate sterile exchange of the card between a customer and store clerk at a point of sale.

The present disclosure includes various embodiments of apparatuses, kits, and methods for sanitizing e.g., disinfecting various objects, such as, for example, credit cards, cell phones, writing instruments, stylus, etc.

Some embodiments of the present sanitizing device comprise: a housing having a front surface and a back surface, the housing defining a front opening through the front surface, a back opening through the back surface, and a chamber in fluid communication with the front opening and the back opening, the housing configured to open to permit a user to access the chamber; and one or more fluid-permeable sponge applicators having a first surface, a second surface, and a length extending between the front surface and the back surface of the chamber, the housing defining a passage extending along the sponge applicator and configured to removably receive and pass an inserted object along an exposed surface of the one or more sponge applicators in the chamber e.g., when the housing is closed. The passage may be substantially aligned with the front opening and the back opening of the housing so that the object can be passed bi-directionally through the front opening, the passage, and the back opening such that when passed through the passage the sponge applicator contacts a substantial portion of one or both faces of the object passed therethrough.

In some embodiments, the passage has a height and a width configured to readily receive an object therein. Accordingly, the height and width of the passage varies according to different objects being sanitized by the sanitizing device. In some embodiments, the sponge applicator extends the length of the passage. In some embodiments, the sponge applicator is layered to receive an object between two layers. In some embodiments, layers of a sponge applicator are joined along a portion of a periphery to form a sponge pocket/passage. In some embodiments, the first surface and the second surface and/or lateral sides of the sponge applicator are configured to be fluid-impermeable to help retain sanitizer fluid therein. In some embodiments, the first surface and the second surface and/or lateral sides of the sponge applicator are coated with a fluid-impermeable material. In some embodiments, an outer surface of the sponge applicator not configured for object contact is coated with the fluid-impermeable material.

In some embodiments, the housing is configured to be removably coupled to a mount. In some embodiments, the housing comprises a first housing member and a second housing member and is configured to be open by separating an upper portion of the first housing member from a lower portion of the second housing member (e.g., body). The housing may be weighted or configured to be removably adhered to a surface or coupled to the mount or to a point-of-sale device.

In some embodiments, the housing comprises a first housing member and a second housing member, and where the housing is configured to open by separating an upper portion of the first housing member from a lower portion of the second housing member. In some embodiments, the first housing member snap-fits or latches to the second housing member. In some embodiments, the first housing member is hinged or pivotally coupled to the second housing member. In some embodiments, where the second housing member is configured to be coupled to the first housing member to define a chamber and configured to be separated from the first housing member to permit a user to access the chamber, e.g., to replace or refresh the sponge applicator.

Some embodiments further comprise: a sanitizer fluid absorbed in the sponge applicator. In some embodiments, the sanitizer fluid is a liquid. In some embodiments, the sanitizer fluid is a gel. In some embodiments, the sanitizer fluid comprises one or more ingredients selected from the group consisting of: alcohols, ethanol, isopropanol, aldehydes, oxidizing agents, acids, phenolics, ammoniums, and chlorine.

In some embodiments, the sanitizing device is configured such that if the sponge applicator is disposed in the chamber and an object is passed in either direction through the front opening, the passage, and the back opening, the sanitizer fluid is applied to the object by the sponge applicator without any external pumping mechanism. In some embodiments, the front opening and the back opening of the housing are configured as slots to allow passage for the object along the sponge applicator.

In some applications, the present method comprises providing a sanitizing device that comprises a housing, a sponge applicator, and a sanitizer fluid absorbed in the sponge applicator; and passing an object through a passage in the housing and along the sponge applicator such that a portion of the sanitizer fluid is disposed on the object.

Some embodiments of the present sanitizer kit comprise: a housing having a front surface and a back surface, the housing defining a front opening through the front surface, a back opening through the back surface, and defining a chamber for retaining a sponge applicator in fluid communication with a sanitizer fluid through a reservoir, the housing configured to open to permit a user to access the chamber; and one or more fluid-permeable sponge applicators having a first surface, a second surface, and a length extending between the first surface and the second surface, the sponge applicator defining a broad surface/face extending between the first surface and the second surface of the sponge applicator; where the housing is configured to removably receive the sponge applicator in the chamber (e.g., when the housing is closed) such that the passage is substantially aligned with the front opening and the back opening of the housing so that an object can be passed in either direction through the front opening, the passage, and the back opening such that when passed through the passage the sponge applicator(s) contacts at least one face of the object; and where the housing is configured to receive one or more sponge applicators.

Some embodiments of the present sanitizing device comprise: a housing having a body with a front surface and a back surface, the housing defining a front opening through the front surface, and an elongated slot portion extending from the back surface of the body, the body defining a chamber; and one or more fluid-permeable sponge applicator(s) disposed within the chamber and having a first surface, a second surface, and a length and broad face extending between the front surface and the back surface; where the housing is configured to removably receive the sponge applicator(s) in the chamber such that an object can be passed in either direction through the front opening, along the sponge applicator(s), and through the back opening. In some embodiments, the sanitizing device is configured such that if the object is disposed in the housing with a portion of the object extending into the housing, resistance from the sponge applicator may resist removal of the object from the housing to prevent it from falling out of either of the front opening or the back opening.

In some embodiments, the housing has a first housing member and a second housing member configured to be coupled to the first housing member to define the chamber and configured to be separated from the first housing member to permit a user to access the chamber. In some embodiments, the dimensions of the front opening and the back opening are selected to readily allow insertion of an object.

In some embodiments, the sanitizing device is configured to be coupled to a point-of-sale (POS) device. In some embodiments, the housing is configured to be removably coupled to the POS device. In some embodiments, the housing is integrated with the POS device.

Any embodiment of any of the present methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Another advantage of the present disclosure is the various possibilities for the placement of the sanitizing device. For example, in some embodiments, the sanitizing device may be positioned on a sale check out surface or even coupled to or integrated with a POS device.

The sanitizing device in accordance with the present disclosure comprises a housing having a front surface with a front opening and a back surface with a back opening. The housing comprises a first housing member, a second housing member, and a chamber disposed therebetween. The chamber is configured to removably receive a fluid-permeable sponge applicator. The sponge applicator receives a sanitizer fluid from a reservoir configured to be disposed above the sponge applicator. The sponge applicator absorbs the received sanitizer fluid. When an object is inserted through the front opening of the front surface into the sanitizing device. The object passes through the passage of the sponge applicator to thereby sanitizing the object. The sanitized object is then retrieved through the back opening of the back surface of the sanitizing device.

In one embodiment, the sanitizing device comprises a connector mechanism which is configured to couple the first housing member and the second housing member. The connector mechanism is selected from a group consisting of snap fit, latch, and hinges.

The present disclosure further discloses a sanitizer kit. The sanitizer kit in accordance with the present disclosure comprises a housing having a front surface and a back surface. The housing further comprises a first housing member, a second housing member and a chamber. The chamber is disposed between the first housing member and the second housing member when they are coupled to each other. The front surface of the housing comprises a front opening. The chamber is enabled to removably receive one or more sponge applicators sealed in one or more capsules. The one or more sponge applicators are preloaded with a sanitizer fluid.

The sanitizer kit is configured to be used to sanitize one or more objects selected from the group consisting of a card, a mobile phone, keys and a writing instrument.

The present disclosure discloses a method for sanitizing an object. The method comprises the steps of providing a sanitizing device comprising: a housing having a front surface and a back surface, and a reservoir, the housing comprising a first housing member and a second housing member connected with a connector mechanism, the first housing member and the second housing member defining a chamber to receive a sponge applicator, the sponge applicator having a sanitizer fluid absorbed therewithin; where the sponge applicator is disposed in the chamber of the housing with a broad face substantially aligned with the front opening and the back opening of the housing so that an object can be passed sequentially through the front opening, along the broad face, and through the back opening such that the sponge applicator contacts the face of the object; and passing the object adjacent and in contact with the sponge applicator such that a portion of the sanitizer fluid is disposed on the object.

### BRIEF DESCRIPTION OF THE DRAWINGS

A more complete understanding of the present invention may be derived by referring to the detailed description and claims when considered in connection with the Figures, wherein like reference numerals refer to similar elements throughout the Figures, and

FIG. 1 illustrates a perspective view of a sanitizing device according to one embodiment of the present disclosure;

FIG. 2 illustrates an exploded perspective view of the sanitizing device of FIG. 1;

FIG. 3 illustrates a front view of the sanitizing device of FIG. 1;

FIG. 4 illustrates a side view of the sanitizing device of FIG. 1;

FIG. 5 illustrates a top view of the sanitizing device of FIG. 1;

FIG. 6 illustrates a bottom view of the sanitizing device of FIG. 1;

FIG. 7 illustrates a perspective front view of the second housing member of the sanitizing device of FIG. 1; and

FIG. 8 illustrates a side view of a second housing member of the sanitizing device of FIG. 1;

FIG. 9 illustrates a top view of a reservoir received within a chamber of the sanitizing device of FIG. 1;

FIG. 10 illustrates a perspective view of the reservoir of FIG. 2;

FIG. 11 illustrates a top perspective view of a first housing member of the sanitizing device of FIG. 1;

FIG. 12 illustrates a front view of the first housing member of FIG. 11;

FIG. 13 illustrates a side view of the first housing member of FIG. 11;

FIG. 14 illustrates an exemplary perspective view of the sanitizing device of FIG. 1, in use with a credit card passing through the housing and sponge applicator;

FIG. 15 illustrates a side view of the sanitizing device of FIG. 14; and

FIG. 16 illustrates a perspective side view of the sanitizing device of FIG. 14.

### LIST OF REFERENCE NUMERALS

Sanitizing device—21

Housing—23

First housing member—25

Second housing member—27

Front opening—33

Back opening—35

Front surface—29

Back surface—31

Removable lid—37

Upper connectors—39

Lower connectors—41

Reservoir—43

Sponge applicator—45

First surface of sponge applicator—47

Second surface of sponge applicator—49

Broad face—51

Chamber—53

Card—55

### DETAILED DESCRIPTION

The following description is of exemplary embodiments of the invention only, and is not intended to limit the scope, applicability or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms and the like and still fall within the scope of the present invention. Thus, the detailed description herein is presented for purposes of illustration only and not of limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

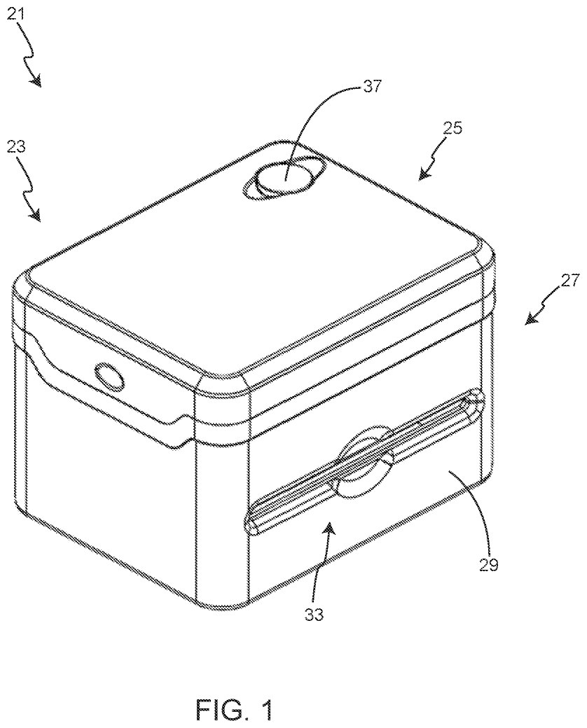

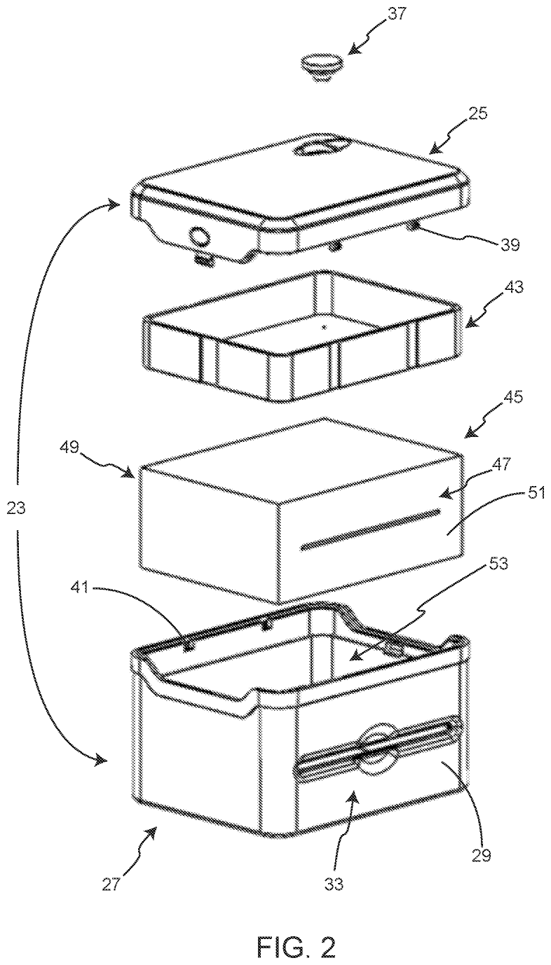

According to a first aspect of the present disclosure, a sanitizing device 21 is disclosed. Reference is made to FIGS. 1-16 that illustrate various views and components of the sanitizing device 21. The sanitizing device 21 comprises a housing 23 and a reservoir 43. The housing 23 has a front surface 29 having a front opening 33 and a back surface 31 having a back opening 35. The housing 23 comprises a first housing member 25, a second housing member 27, and a chamber 53 defined by the first housing member 25 and the second housing member 27. The first housing member 25 and the second housing member 27 are coupled to each other using a connector mechanism 39, 41 (seen in FIG. 2) thereby forming the housing 23. The chamber 53 is configured to removably receive a fluid-permeable sponge applicator 45. The sponge applicator 45 is configured to absorb a sanitizer fluid for sanitization of an object.

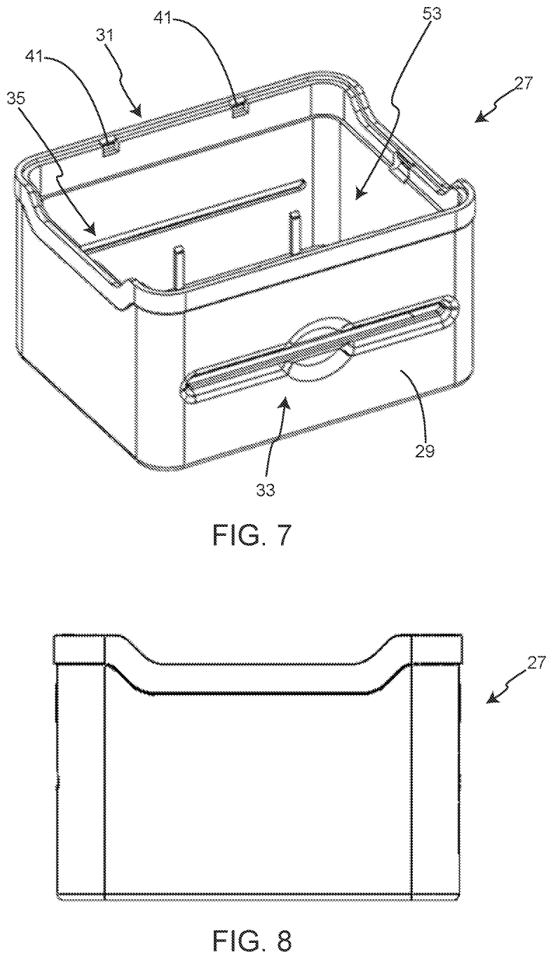

In some embodiments, the back surface 31 of the housing 23 comprises a back opening 35, best seen in FIG. 7, to facilitate removal of an inserted object after sanitization. Hence, in such embodiments, an object is inserted into the front opening 33 and removed from the back opening 35 after passing through the sponge applicator 45, in other words, after getting sanitized. It is to be noted that the insertion of the object may be carried out from the back opening 35 and removal of the sanitized object may be done from the front opening 33 as well without departing from the scope of the present disclosure. It is to be further noted that the front opening 33 and the back opening 35 may be configured to receive an object of a specific shape. Further, the front opening 33 and the back opening 35 may be configured to have various shapes based on shapes of objects which are to be sanitized by the sanitizing device 21.

The sponge applicator 45 is configured to be removably placed inside the chamber 53. The first housing member 25 and the second housing member 27 may be detached and separated by means of a connector mechanism for replacing or adjusting the sponge applicator 45. In some embodiments, the first housing member 25 snap-fits or latches to the second housing member 27. In some embodiments, the first housing member 25 is hinged or pivotally coupled to the second housing member 27. The first housing member 25 and the second housing member 27 are configured to be detached and separated to permit a user to access the chamber 53, e.g., to replace or refresh the sponge applicator 45.

The sponge applicator 45 comprises a first surface 47 defining a broad face 51, and a second surface 49. When placed in the chamber 53, the first surface 47 of the sponge applicator 45 is configured to be adjacent to the front surface 29 of the housing 23, in particular, of the second housing member 27 and the second surface 49 of the sponge applicator 45 is configured to be adjacent to the back surface 31 of the housing 23, in particular, of the second housing member 27.

In some embodiments, the sponge applicator 45 is layered from the first surface 47 to the second surface 49 to receive an object between two layers. In such embodiments, the sponge applicator 45 is slit such that a layer of the slit coincides with the first opening and the second opening of the housing 23. In some embodiments, layers of the sponge applicator 45 are joined along a portion of a periphery to form a sponge pocket/passage. In some embodiments, first and second ends and/or lateral sides of the sponge applicator 45 are configured to be fluid-impermeable to help retain sanitizer fluid therein. In some embodiments, the first and second ends and/or lateral sides of the sponge applicator 45 are coated with a fluid-impermeable material. In some embodiments, an outer surface of the sponge applicator 45 not configured for the object contact is coated with a fluid impermeable material.

Fluid-impermeable material comprises, for example, one or more materials selected from the group consisting of paints, lacquers, polymers, plastics, and the like. Another example of a fluid-impermeable material is a polyurethane solution (e.g., with a vinyl solvent base), such as, for example, PDC® F-874 MURACULON, available from PLASTI DIP INTERNATIONAL, USA). In this way, the sponge applicator 45 is configured to resist evaporation of sanitizer fluid from the sponge applicator 45. The sponge applicator 45 is further configured to remove or absorb excess sanitizer fluid from the object as the object is passed out of the sanitizing device 21. In some embodiments, the sanitizing device 21 further comprises flaps disposed within the housing 23 adjacent to the front opening 33 and the back opening 35 to reduce evaporation of the sanitizer fluid.

The reservoir 43 is configured to be placed above the sponge applicator 45. The reservoir 43 is configured to contain the sanitizer fluid which is absorbed by the sponge applicator 45 to sanitize the object. For the same, the reservoir 43 comprises at least one liquid port, through which the sanitizer fluid passes to the sponge applicator 45. In some embodiments, the sanitizer fluid is liquid. In other embodiments, the sanitizer fluid comprises a gel. The gel in such embodiments is configured or formulated to resist evaporation.

The sanitizer fluid comprises, for example, one or more ingredients selected from the group consisting of: alcohols, ethanol, isopropanol, aldehydes, oxidizing agents, acids, phenolics, ammoniums, and chlorine. One example of the sanitizer fluid suitable for some of the present embodiments is benzalkonium chloride (BZK), such as, for example, a solution with equal to, less than, or between any of: 0.1, 0.13, 0.15, 0.2, and/or 0.26 percent by weight of benzalkonium chloride. The sanitizing device 21 of the present disclosure may not comprise a pump mechanism to wet the sponge applicator 45 with the sanitizer fluid, and instead, is configured with the reservoir 43 disposed above the sponge applicator 45 and with a liquid port to allow the sanitizer fluid to pass from the reservoir 43 to wet or saturate the sponge applicator 45. In an embodiment, the first housing member 25 comprises a removable lid 37 to refill the reservoir 43 with the sanitizer fluid. Hence, the sanitizer fluid can be applied to the object by the sponge applicator 45 without any external pumping mechanism.

FIG. 2 illustrates the exploded perspective view of the sanitizing device 21 as illustrated in FIG. 1. As shown in FIG. 2, the removable lid 37 of the first housing member 25 is configured to be removed to fill the sanitizer fluid in the reservoir 43 which is placed underneath the first housing member 25. The sponge applicator 45 is placed below the reservoir 43 and is in the fluid communication with the reservoir 43. Further, the sponge applicator 45 comprises a slit at the first surface 47 for insertion of the object to be sanitized. The slit extends throughout a length of the sponge applicator 45 from the first surface 47 to the second surface 49. The slit is designed in such a way that the slit at the first surface 47 corresponds to the front opening 33 of the housing 23 and at the second surface 49, the slit corresponds to the back opening 35 of the housing 23. The reservoir 43 and sponge applicator 45 are placed inside the chamber 53 defined by the first housing member 25 and the second housing member 27. The sponge applicator 45, being fluid permeable, absorbs the sanitizer fluid from the reservoir 43 through at least one liquid port of the reservoir 43. Hence, when an object is inserted through the front opening 33, the object passes through the slit of the sponge applicator 45 to get sanitized by the absorbed sanitizer fluid in the sponge applicator 45.

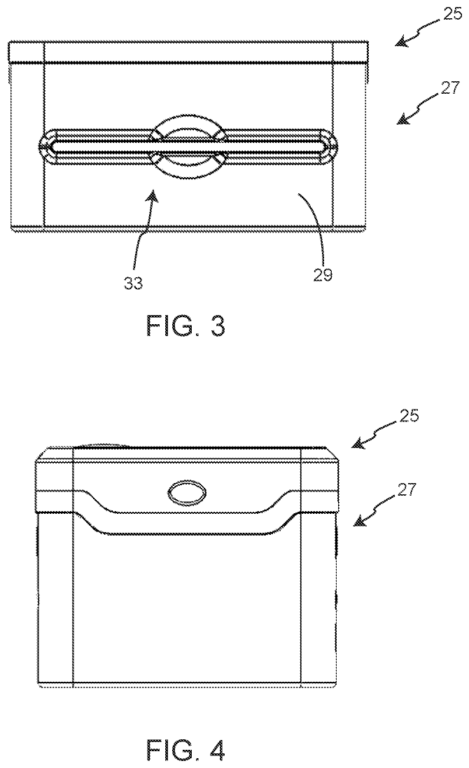

FIG. 3 illustrates an exemplary front view of the sanitizing device 21 in accordance with the present disclosure. The sanitizing device 21 is in a closed (assembled) state in which the first housing member 25 and the second housing member 27 are connected via a connector mechanism. In such embodiment, the sponge applicator 45 may be placed inside the chamber 53 formed by the first housing member 25 and the second housing member 27. FIG. 4 illustrates a side view of the sanitizing device 21 in accordance with the present disclosure in which the first housing member 25 and the second housing member 27 are assembled by means of the connector mechanism 39, 41 (not visible).

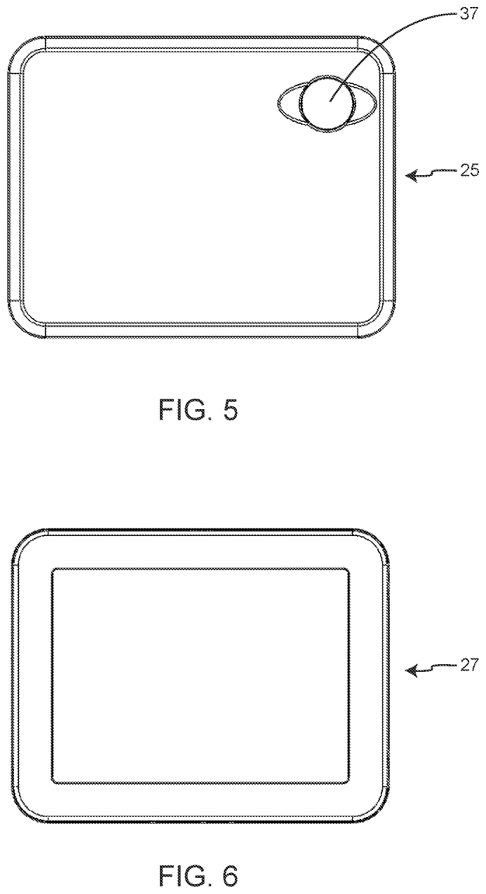

FIG. 5 and FIG. 6 illustrate a top and a bottom view respectively of the sanitizing device 21 in accordance with the present disclosure. The top view as seen in FIG. 5, shows the removable lid 37 attached at the first housing member 25. The removable lid 37 is configured to be detached from the first housing member 25 for filling the reservoir 43 of the sanitizer fluid. FIG. 6 shows the bottom view of the sanitizing device 21 which acts as a base for providing support to the sanitizing device 21. It is appreciated that although the sanitizing device 21 having a rectangular shape is illustrated, the sanitizing device 21 may have a different shape without departing from the scope of the present disclosure.

FIG. 7 illustrates a perspective view of the second housing member 27 of the sanitizing device 21. The second housing member 27 is in a detached state from the first housing member 25. The second housing member 27 comprises a plurality of lower connectors 41. The lower connectors 41 are configured to engage with corresponding plurality of upper connectors 39 provided at the first housing member 25. FIG. 8 illustrates a side view of the second housing member 27 of the sanitizing device 21 of the present disclosure, the second housing member 27 being detached from the first housing member 25.

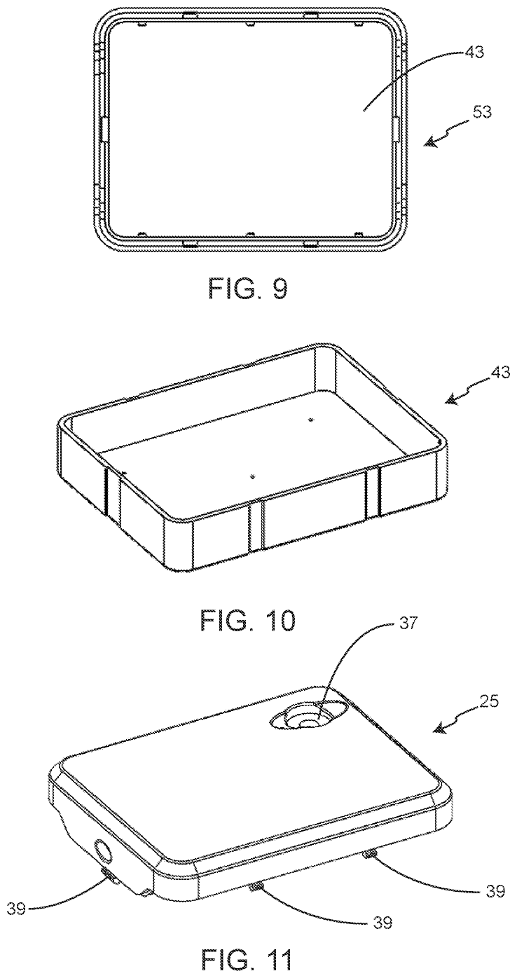

FIG. 9 illustrates a top view of reservoir 43 being positioned within chamber 53. FIG. 10 illustrates a perspective view of reservoir 43 in accordance with the present disclosure.

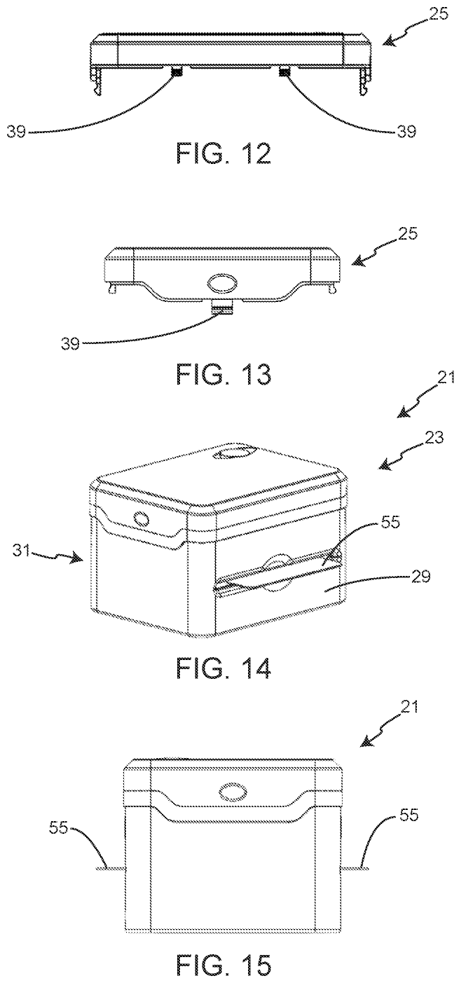

FIGS. 11-13 illustrate different views of the first housing member 25 of the housing 23 of the sanitizing device 21 of the present disclosure. FIG. 11 illustrates a perspective view of the first housing member 25 showing the removable lid 37 configured to be removed to fill the reservoir 43 with the sanitizer fluid and a plurality of upper connectors 39 of the connector mechanism configured to be attached to corresponding plurality of lower connectors 41 provided at the second housing member 27 in order to form the chamber 53 therewithin. FIG. 12 and FIG. 13 illustrate a front view and a side view of the first housing member 25, respectively, showing a plurality of upper connectors 39 of connector mechanism to be connected with the corresponding lower connectors 41.

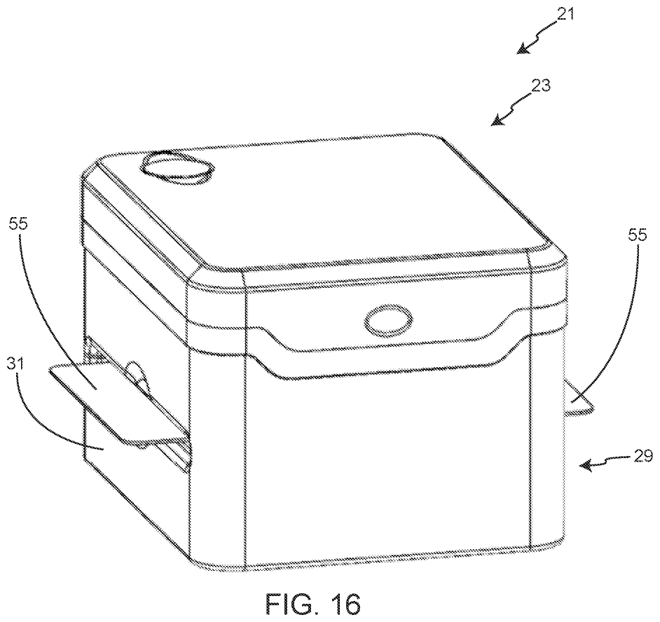

FIGS. 14-16 illustrate an exemplary sanitizing device 21 in accordance with the present disclosure showing a card 55 as an object getting sanitized. As shown in FIG. 14, the card 55 is inserted into the housing 23 from the front opening 33. The card 55 is then passed through the sponge applicator 45, thereby getting sanitized. FIGS. 15-16 show the card 55 at the back opening 35 for removal after the sanitization.

The present disclosure relates to sanitization of an object. Some non-limiting examples of the object are a card 55, a mobile phone, a writing instrument, and the like. The present disclosure explains different features, figures, and embodiments of a sanitizing device 21 for sanitizing a card 55. However, it is evident that the sanitizing device 21 may be used for sanitizing other objects as well. The mere exemplary explanation of the device hereinbelow does not limit or restrict the scope of the invention and the application of the device.

In an embodiment of the present disclosure, the sanitizing device 21 is configured to be coupled to a tray at the back surface 31 of the sanitizing device 21 enabling it to capture the objects after they get sanitized. In another embodiment of the present disclosure, when the object to be sanitized is inserted at the front opening 33 of the sanitizing device 21, the object automatically gets retrieved at the back opening 35 after sanitization, with effect from the slippery nature of the sponge applicator 45 and the sanitizer fluid. In yet another embodiment of the present disclosure, the object to be sanitized is inserted at front opening 33 of the sanitizing device 21 to be retrieved manually at the back opening 35. In yet another embodiment of the present disclosure, the object to be sanitized can be inserted either from the front opening 33 or the back opening 35, to be retrieved at the respective back opening 35 or the front opening 33.

In some embodiments, the sanitizing device is configured to be coupled to a point-of-sale (POS) device. In some embodiments, the housing 23 is configured to be removably coupled to a POS device. In some embodiments, the housing 23 is integrated with a POS device.

According to a second aspect of the present disclosure a sanitizer kit is disclosed for sanitizing an object. The sanitizer kit comprises a housing 23 having a front surface 29 and a back surface 31, and one or more sponge applicators 45 sealed in one or more capsules. It is to be noted that the housing 23 and the sponge applicator 45 of the kit corresponds to the housing 23 and the sponge applicator 45 of the sanitizing device 21 as explained hereinabove with reference to FIGS. 1-16. Accordingly, all features of the housing 23 of the sanitizing device 21 are part of the housing 23 of the sanitizer kit. Accordingly, the housing 23 comprises a first housing member 25 and a second housing member 27. The first housing member 25 and the second housing member 27 are configured to be connected with a connector mechanism. The first housing member 25 and the second housing member 27 defines a chamber 53 therewithin to removably receive the one or more sponge applicators 45. It is further to be noted that the sponge applicator 45 of the sanitizer kit bears similar characteristics of the sponge applicator 45 of the sanitizing device 21 disclosed hereinabove.

The one or more sponge applicators 45 are fluid-permeable and contain sanitizer fluid therewithin. Each of the one or more capsules, in which a corresponding sponge applicator 45 is sealed, is impermeable to fluid and/or gas to prevent the sanitizer fluid from evaporating out of the sponge applicator 45 sealed therewithin.

According to a third aspect of the present disclosure, a method for sanitizing an object is disclosed. It is to be noted that the method is performed on the sanitizing device 21 of FIG. 1 as disclosed hereinabove and hence, all the features of the sanitizing device 21 are incorporated. Accordingly, the method in accordance with the present disclosure comprises: providing a sanitizing device 21 comprising: housing 23, sponge applicator 45, and a sanitizer fluid absorbed in the sponge applicator 45; where the sponge applicator 45 is disposed in a chamber 53 of the housing 23 with a broad face 51 substantially aligned with a front opening 33 and a back opening 35 of the housing 23 so that an object can be passed sequentially through the front opening 33, along the broad face 51, and through the back opening 35 such that the sponge applicator 45 contacts the face of the object. The method further comprises passing the object adjacent and in contact with the sponge applicator 45 such that a portion of the sanitizer fluid is disposed on the object. The object is configured to be passed through the sanitizing device 21 from the front opening 33 or the back opening 35. Some non-limiting examples of the object are cards 55, mobile phones, keys, or other similar devices now known or hereafter developed.

Any of the present devices, kits, and/or methods can be configured to have any of the features described and/or depicted for any of the other devices, kits, and/or methods. For example, the first end and/or second end of the sponge applicator of any of the devices can be configured to be fluid-impermeable.

The various illustrative embodiments of devices, kits, and methods described herein are not intended to be limited to the particular forms disclosed. Rather, they include all modifications, equivalents, and alternatives falling within the scope of the claims.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

1. A sanitizing device comprising:
a housing having a front surface and a back surface, the front surface comprising a front opening, the housing comprising a first housing member, a second housing member, and a chamber defined by the first housing member and the second housing member;
a fluid-permeable sponge applicator configured to be disposed within the chamber;
a reservoir configured to be disposed above the sponge applicator, the reservoir configured to hold sanitizer fluid for absorption by the sponge applicator;
wherein the front opening is configured to allow insertion of an object to be sanitized into the housing and towards the back surface while passing through the sponge applicator, to thereby sanitize the exterior of the object.

2. The device of claim 1, wherein the back surface comprises a back opening configured to facilitate removal of the object after being sanitized by passing through the sponge applicator.

3. The device of claim 2, wherein the back opening is further configured to facilitate insertion of the object to get sanitized.

4. The device of claim 1, wherein the first housing member and the second housing member are configured to be coupled using a connector mechanism.

5. The device of claim 1, wherein the first housing member and the second housing member are configured to be detached to replace the sponge applicator within the chamber.

6. The device of claim 1, wherein the sponge applicator comprises a first surface adjacent to the front surface of the housing and a second surface adjacent to the back surface of the housing, the sponge applicator being configured to be layered, slit, or partially severed between the first surface and the second surface to allow the object to pass therethrough to be sanitized.

7. The device of claim 1, wherein the reservoir comprises at least one liquid port to allow the sanitizer fluid to pass from the reservoir to the sponge applicator.

8. The device of claim 1, wherein the first housing member comprises a removable lid configured to be removed to refill the sanitizer fluid in the reservoir.

9. The device of claim 4, wherein the connector mechanism is selected from a group consisting of snap fit, latch, and hinges.

10. The device of claim 6, wherein the first surface and the second surface of the sponge applicator are coated with a partially fluid-impermeable material to resist evaporation of sanitizer fluid.

11. The device of claim 10, wherein the fluid-impermeable material comprises one or more materials selected from the group consisting of paints, lacquers, polymers, plastics.

12. The device of claim 1, wherein the sanitizer fluid comprises one or more ingredients selected from the group consisting of: alcohols, ethanol, isopropanol, aldehydes, oxidizing agents, acids, phenolics, ammoniums, and chlorine.

13. The device of claim 2, wherein the device comprises flaps disposed within the housing adjacent to the front opening and the back opening to reduce evaporation of the sanitizer fluid.

14. The device of claim 1, wherein the object to be sanitized is selected from the group consisting of a card, a mobile phone, and a writing instrument.

15. The device of claim 1, wherein the device is configured to be removably adhered to a surface or coupled to a point-of-sale (POS) device.

16. The device of claim 1, wherein the sponge applicator is configured to have a rectangular cross-section.

17. A sanitizer kit, the kit comprising:
a housing having a front surface and a back surface, the housing comprising a first housing member, a second housing member, and a chamber defined by the first housing member and the second housing member, the front surface comprising a front opening; and
one or more sponge applicators sealed in one or more capsules, each of the one or more sponge applicators configured to be preloaded with a sanitizer fluid, and further configured to be removably placed within the chamber of the housing.

18. The kit of claim 17, wherein each of the one or more capsules is impermeable to fluid and/or gas to prevent the sanitizer fluid from evaporating out of the sponge applicator sealed therewithin.

19. The kit of claim 17, wherein the kit is configured to be used to sanitize one or more objects selected from the group consisting of a card, a mobile phone, a writing instrument, and keys.

20. A method for sanitizing an object, the method comprising:
providing a sanitizing device comprising: a housing having a front surface and a back surface, and a reservoir, the housing comprising a first housing member and a second housing member connected with a connector mechanism, the first housing member and the second housing member defining a chamber to receive a sponge applicator, the sponge applicator having a sanitizer fluid absorbed therewithin; wherein the sponge applicator is disposed in the chamber of the housing with a broad face substantially aligned with the front opening and the back opening of the housing so that an object can be passed sequentially through the front opening, along the broad face, and through the back opening such that the sponge applicator contacts the face of the object; and
passing the object adjacent and in contact with the sponge applicator such that a portion of the sanitizer fluid is disposed on the object.
